(19) United States
(12) Patent Application Publication
(10) Pub. No.: US 2003/0082249 A1
(43) Pub. Date: May 1, 2003

(54) Compositions containing capsaicin and its derivatives, and their use in treating mucositis

(75) Inventor: Gary Gordon, Highland Park, IL (US)

Correspondence Address: KATTEN MUCHIN ZAVIS, Attention: Patent Administrator, Suite 1600, 525 West Monroe Street, Chicago, IL, 60661-3639, US

(73) Assignee: Ovation Pharmaceuticals

(21) Appl. No.: 10/004,216

(22) Filed: Oct. 26, 2001

(51) Int. Cl. A61K 35/78; A61K 9/68; A61K 31/16

(52) U.S. Cl. 424/760; 424/48; 514/625

(57) ABSTRACT

A composition for use in treating or preventing mucositis, and/or xerostomia, including capsaicin or capsaicin derivative, and one or more additional compounds useful in treating mucositis and/or xerostomia, wherein the composition is provided in an oral delivery vehicle. The invention also relates to a method of treating or preventing mucositis and/or xerostomia, and a method for preparing such a composition.

### BACKGROUND OF THE INVENTION

1. Field of the Invention

This invention relates to compositions containing capsaicin or a derivative thereof, and also includes one or more additional compounds useful in treating mucositis. The compositions may be used in methods of treating mucositis. The composition preferably is administered orally. The method and composition are highly effective in relieving mucositis caused by a variety of underlying pathologies.

2. Related Art

Mucositis is a painful disorder characterized by redness, painful ulcerations, and swelling in the mouth and esophagus. In more severe cases, mucositis can be extremely painful, preventing the patient from eating, drinking or even swallowing their own saliva. This condition can necessitate hospitalization for treatment of pain, including the use of parenteral narcotics, as well as fluids and/or total parenteral nutrition to treat dehydration and meet the heightened nutritional requirements imposed by cancer therapy. The destruction of the protective mucous membrane also places the patient at a serious risk of local and systemic infection. Organisms frequently involved include bacteria, viruses or fungi and can severely exacerbate the underlying difficulties. Mucositis often limits the amount of chemotherapy and radiation therapy that can be administered to a patient, leading to reductions or delays in treatment that can adversely impact the outcome of the patient's therapy.

It is estimated that mucositis affects up to 40% of patients receiving standard-dose chemotherapy, and 76-100% of patients receiving higher doses of chemotherapy. Mucositis also affects virtually all patients receiving radiation therapy for head and neck cancer, as well as patients receiving radiation along the gastrointestinal tract. Mucositis likely will increase in frequency and severity as more aggressive treatment regimens are used to treat cancers.

There is an unmet need for mucositis therapies that prevent or reduce the severity and duration of symptoms, so that patient's chemotherapy or radiation regimen can be maintained or intensified. Clinicians also desire a therapy that reduces hospitalization, narcotic use, or the need for nutritional support.

A factor that can enhance the development of mucositis and can as well result from cancer treatment is xerostomia, or dry mouth. Xerostomia can result from decreased saliva production within the salivary glands (i.e., parotid, submandibular, sublingual, and accessory salivary glands) and/or diminished secretion of saliva from the glands following autonomic stimulation. It can also result from alteration of the composition of the saliva. Decreased saliva prevents adequate lubrication of the oral cavity resulting in an uncomfortable oral sensation, difficulty in speaking and swallowing. Severe cracking of the tongue can also result. Xerostomia can also result in an increase in tooth decay and an increased incidence of oral infections which can in turn cause or exacerbate mucositis. There are many causes of xerostomia, which is often associated with mucositis, including medical therapies (e.g., radiotherapy to the head and neck; drugs including tricyclic-antidepressants, antihistamines, decongestants, benzodiazepines, phenothiazine derivatives, anti-Parkinson's medications, narcotic analgesics, appetite suppressants, anticholinergics, anti-spasmodics, antiemetics, antidiarrheals, anticonvulsants, antihypertensives, and psychotropic agents, and antirejection drugs), surgical and traumatic etiologies, systemic disorders (e.g., autonomic dysfunction, CNS conditions such as Alzheimer's disease, the autoimmune rheumatic disease Sjogren's Syndrome, graft-versus-host-disease, sarcoidosis, and amyloidosis), dehydration (e.g., due to insufficient water intake, loss of water through the skin due to fever, excessive sweating or burns, loss of blood, diarrhea, renal insufficiency and subsequent water loss due to diabetes, protein malnutrition), elevated stress and with certain endocrine diseases. It can also result from traumatic injury to the head or neck or from head or neck surgery. Destruction of the salivary glands and/or its controlling autonomic nerves can also result in xerostomia. The elderly appear to be more affected by xerostomia, likely due to increased use of xerostomia-inducing medications or a higher incidence of certain systemic disorders that may cause xerostomia and in fact the elderly generally experience more severe mucositis than younger individuals in response to cancer treatment.

Treatment of xerostomia includes treatment of the underlying etiology if possible and the use of compounds or manipulations that increase salivary flow as well agents that enhance the providing compounds useful in preventing the severe tooth decay that can be caused by xerostomia. Stimulation of salivary flow through pharmacologic, mechanical or other means provides the most efficacious relief of symptoms. A regular chewing action provides a mechanical stimulus for salivary production. Chemical stimulation of salivary flow may be achieved utilizing substances such as citric acid, sour and sugarless candies, or lozenges. In addition, pilocarpine has been shown to be effective in stimulating salivary glands of patients experiencing xerostomia due to radiotherapy. The use of pilocarpine to stimulate saliva production is described for example in U.S. Pat. No. 5,571,528 (incorporated herein by reference). However, pilocarpine treatment is often associated with side effects involving heart, blood pressure, digestion and profuse sweating. Artificial saliva may also be used to alleviate the symptoms if other treatments are ineffective.

However, none of the methods or compositions discussed above address the problem of treating mucositis or xerostomia by affecting one or more of its multiple etiologies by administering capsaicin or a capsaicin derivative such as civamide in conjunction with an additional compound, or compounds, such as those described herein.

### SUMMARY OF THE INVENTION

While relief from mucositis is partially achievable using single agents, combinations that impact multiple etiologies provide more effective and sustained relief. In one of its aspects, the nature of this invention is directed to the use of a composition including capsaicin or a capsaicin derivative (capsaicinoid), in combination with one or more additional compounds useful in relieving mucositis. This invention also relates to a composition including capsaicin or a capsaicin derivative (capsaicinoid), and a method of treating xerostomia by inducing salivation by applying such a composition to the mucosal tissues of the mouth. Such formulations can take the form of preparations that are expectorated following application to the mucosa or swallowed after application.

The methods and compositions of this invention address the need in the art for an effective anti-mucositic treatment, as set forth above. More specifically, and according to one aspect of this invention, a composition for use in treating mucositis, comprises capsaicin or capsaicin derivative, and one or more additional compounds useful in treating mucositis, wherein the composition is provided in an oral delivery vehicle.

According to yet another aspect of this invention, a method for treating mucositis comprises the steps of administering to a patient a therapeutically effective amount of a composition comprising a capsaicin or capsaicin derivative and one or more additional compounds useful in treating mucositis, and repeating administration as necessary to relieve the mucositis.

According to another aspect of this invention, a method for preparing a composition for use in treating mucositis comprises the step of providing a compound comprising capsaicin or a capsaicin derivative and one or more additional compounds, which may be selected from the group consisting of antifungal compounds, antibacterial compounds, antiviral compounds, anesthetic compounds, analgesic compounds, anti-inflammatory compounds, cytoprotective compounds, anti-histaminic compounds, anti-mucositic compounds, and anti-xerostomic compounds, and the step of combining said compound or compounds with an oral delivery system, thereby forming an oral preparation.

It will be apparent to those skilled in the art that only the preferred embodiments have been described by way of exemplification and that there are various modifications which fall within the scope of this invention. These and other aspects of the invention will be discussed in greater detail below.

### DETAILED DESCRIPTION OF THE PREFERRED EMBODIMENTS

While the present invention will be described primarily with respect to a method and composition for treating mucositis and/or xerostomia, it is to be understood that the features thereof will find applicability to other areas, such as the treatment of other disorders of the mucosa. The term “mucosiltis” as used herein is meant to refer to the condition characterized by painful lesions that form in the oral mucosa, and severe dry mouth or xerostomia, often as a result of radiographic treatment of the head and/or neck or other etiologies as outlined above. The term “xerostomia” as used herein is meant to refer to dryness of the mouth due to decreased saliva production, which may be mild or severe. Xerostomia may be caused by any of a number of etiologies.

Briefly, this invention relates to a method and composition for relieving mucositis using a combination of compounds, in order to provide more effective and sustained relief. The primary compound responsible for the anti-mucositic or anti-xerostomic action of the compositions of this invention is a capsaicinoid. The term capsaicin derivative and capsaicinoid as used herein are interchangeable and generally refer to capsaicin analogs. Among the capsaicinoids useful in the practice of the present invention are capsaicin, the N-phenylmethylalkenamide capsaicin derivatives; dihydrocapsaicin; norhydrocapsaicin; nordihydrocapsaicin; homocapsaicin; homohydrocapsaicin; homodihydrocapsaicin; civamide (cis-capsaicin); nonivamide; NE-19550 (N-[4-hydroxy-3-methoxyphenyl)methyl]-9Z-octadecanamide) (olvanil); NE-21610 (N-[(4-(2aminoethoxy)-3-methoxyphenyl)methyl]-9Z-octadecanamide) Sandoz Pharmaceutical Corp, East Hanover, N.J.) (Chemical Abstract Service Registry No. 118 090 178); NE-28345 (N-(9Z-octadecenyl)-3-methoxy-4-hydroxyphenylacetamide) (Chemical Abstract Registry No. 107 512 561) (also known as N-oleyl-homovanillamide); and their analogs and derivatives. (See, e.g., U.S. Pat. No. 5,762,963, which is incorporated herein by reference.) NE-19550, NE-21610, and NE-28345 are discussed in Dray et al., Eur. J. Pharmacol., 181(3):289-293 (Netherlands 1990) and Brand et al., Agents Actions, 31(3-4): 329-340 (Switzerland 1990). Civamide (cis-capsaicin) comprises a preferred embodiment of the present invention, in part because civamide is believed to deplete substance P more effectively than other capsaicinoids, thereby preventing pain without decreasing sensation or affecting motor coordination. This action is considered beneficial because it allows relief of the pain associated with mucositis without causing a “thick tongue” sensation, and prevents injury and infection caused by inadvertent biting of the tongue or cheeks. The capsaicinoid is provided in an amount ranging from about 0.01% to about 5% by weight, and preferably from about 0.025% to about 0.25% by weight.

In a preferred embodiment of the invention, the capsaicin derivative is civamide. Civamide has been shown to be more effective than capsaicin in depleting substance P thereby preventing the sensation of pain without compromising the sensation of touch or altering motor coordination. This should allow pain relief without the usual sensation of a thick tongue and with a decreased risk of injury/infection due to inadvertent biting of the tongue or cheeks.

To this capsaicinoid, which as stated above, is preferably civamide, one or more additional compounds useful in relieving the mucositic or xerostomic condition is added to alleviate the symptoms of the condition, as well as to minimize any undesirable side effects of capsaicin. These additional agents may include one or more of antibacterial agents, antiviral agents, antifungal agents, anti-inflammatory agents, cytoprotective agents, and various anti-mucositic and anti-xerostomic agents. Additional compounds known to those of skill in the art may be included as carriers, preservatives, excipients, and adjuvants, and these should be selected based on their effectiveness in treating the cause of the mucositis or xerostomia in the particular patient. The composition may be provided in any form suitable for administration to oral mucosa, including, but not limited to, gels, creams, and lozenges. The compositions and methods according to this invention are suitable for use in relieving mucositis and/or xerostomia in animals, and are preferably used to treat mucositis and/or xerostomia in mammals. Particularly preferred compositions and methods are useful in treating mucositis and/or xerostomia in humans.

Antifungal Agents

These compounds are provided in amounts ranging from about 0.1% to about 10% by weight. Suitable antifungals for use in compositions for treatment of mucositis according to this invention include, but are not limited to: amphotericin, butenafine, ciclopirox, clotrimazole (preferably 1% or 10 mg), fluconazole, griseofulvin, itraconazole, ketoconazole, miconazole (preferably 2%), naftifine, nystatin (preferably in an amount equivalent to about 100,000 units/ml), oxiconazole, polymyxin and terbinafine and other relevant antifungal agents as discovered.

Antibacterial Agents

These compounds are provided in amounts ranging from about 0.1% to about 10% by weight. Suitable antibacterials for use in compositions for treatment of mucositis according to this invention include, but are not limited to: hydrogen peroxide, chlorhexidine, other topical anti-infectives, ampicillin or other penicillin derivatives, ciprofloxacin or other cephalosporin derivatives, tobramycin or other aminoglycoside derivatives, polymixin, bacitracin, or combinations such as PTA (combination of polymixin E, tobramycin and amphotericin B), PVP iodine, and other classes of antibiotic agents as they discovered.

Antiviral Agents

These compounds are provided in amounts ranging from about 0.1% to about 10% by weight. Suitable antivirals for use in compositions for treatment of mucositis according to this invention include, but are not limited to: acyclovir (Herpes simplex viruses [HSV]), famciclovir (HSV), penciclovir (HSV), trifluridine (HSV), ganciclovir (cytomegalovirus [CMV] [HSV]), cidofovir (CMV), indinavir (human immunodeficiency virus [HIV]), amprenavir (HIV), lamivudine (HIV), zidovudine (HIV), efavirenz (HIV), saquinavir (HIV), and rimantadine (influenza A [INFLU-A]), amantadine (INFLU-A), oseltamivir (INFLU-A), palivizumab (respiratory syncitial virus [RSV]), the interferons (Hepatitis C and Hepatitis B [HEP-C], [HEP-B]), and other relevant antiviral agents as discovered.

Topical Anesthetic/Analgesics

These compounds are provided in amounts ranging from about 0.5% to about 15% by weight. Suitable anesthetics and analgesics for use in compositions for treatment of mucositis according to this invention include, but are not limited to: benzocaine, lidocaine, prilocaine, dyclonine, cocaine, pramoxine, and other relevant topical anesthetics as discovered.

Anti-Inflammatory Agents

These compounds are provided in amounts ranging from about 0.05% to about 5% by weight. Suitable anti-inflammatory agents for use in compositions for treatment of mucositis according to this invention include, but are not limited to: topical corticosteroids (hydrocortisone, dexamethasone, betamethsone, prednisone, prednisolone, flurandrenolide, clocortolone, triamcinolone, alclometasone), inhibitors of cyclo-oxygenase 1 and 2 (benzdyamine, indomethacin, diclofenac, piroxicam) as well as selective inhibitors of cyclooxygenase 2, (celecoxib, rofecoxib, paracoxib, valdecoxib). Additional agents in this general class include and postaglandin E2, misoprostol, chamomile, and curcumin, other plant derived polyphenols and antioxidants.

Cytoprotective Agents

These compounds are provided in amounts ranging from about 0.1% to about 20% by weight. Suitable cytoprotective agents for use in compositions for treatment of mucositis according to this invention include, but are not limited to: sucralfate, sodium alginate, kaolin-pectin, beta-carotene (pro-vitamin A) and other retinoids (tretinoin, 9-cis-retinoic acid, etc) vitamin E, pentoxifylline, vitamin D and its analogs and antacids, allopurinol, amifostine, glutamine, propantheline, and azelastine.

Agents Altering Cellular Differentiation or Proliferation

These agents are provided in amounts ranging from 0.1 to 5% by weight. Suitable agents include transforming growth factor beta3, GM-CSF, G-CSF and other cytokines that may influence cell growth, differentiation, migration, recruitment or proliferation.

Antihistaminic Agents

These compounds are provided in amounts ranging from about 0.1% to about 5% by weight. Suitable antihistamines for use in compositions for treatment of mucositis according to this invention include H1 and/or H2 blockers, including but not limited to: acrivastine, astemizole, azatadine maleate, azelastine HCI, bromopheniramine maleate, cetirizine HCL, chloropheniramine maleate, cyproheptadine HCl, diphenhydramine, fexofenadine HCl, loratadine, phenindaminie maleate, promethazine HCl, pyrilamine maleate, and terfenadine.

Additional Agents

These compounds are provided in amounts ranging from about 0.1% to about 10% by weight. Suitable agents for use in compositions for treating mucositis and xerostomia according to this invention include, but are not limited to: orally administered fluoride-containing compounds, chamomile, silver nitrate, citric acid and pilocarpine.

In more detail, the capsaicin-containing compositions of this invention for use in treating mucositis and xerostomia may be formulated as creams, aqueous or non-aqueous suspensions, lotions, emulsions, suspensions or emulsions containing micronized particles, gels, foams, aerosols, solids and other suitable vehicles for application to the lips and mucosa, as lozenges or chewing gums, other candy formulations, chilled formulations such as ices, popsicles and as combinations with bandages, patches, bioadhesives, and dressings for local and topical administration, as appropriate for the particular condition and subject being treated.

Formulations and Methods

Effective concentrations of the compositions of this invention, or pharmaceutically acceptable derivatives thereof, are mixed with a pharmaceutical carrier or vehicle suitable for administration to the area to be treated. The compositions are provided in an amount effective for alleviating the mucositic or xerostomic condition for which treatment is contemplated. The concentration of the active compounds in the compositions depend on absorption, inactivation, and excretion rates of the active compound, the dosage schedule, and amount administered as well as other factors known to those of skill in the art.

The dosage of the capsaicin or capsaicinoid (preferably civamide) is from about 0.01% to about 2.5% by weight for antimucositic/antixerostomia purposes when administered to the oral mucosa. The dosage of the additional compound varies depending on the specific compound selected, the individual being treated, and other factors known to those of skill in the art.

The compositions may be administered orally, and a preferred vehicle is a gum, lozenge or a mouth wash. However, pharmaceutical carriers or vehicles suitable for administration of the compounds and for use with the methods provided herein include any such carriers known to those skilled in the art to be suitable for the particular mode of administration. In addition, the composition may be formulated with other active ingredients.

1. Oral Formulations

Typically a therapeutically effective dosage of the composition of this invention for use in treating dry mouth is formulated to contain a concentration of at least about 0.1% by weight, up to about 2.5% by weight or more of capsaicin or a capsaicin derivative. The optimal amount of these compounds may vary depending on the severity of the xerostomia or mucositis and the subject being treated, and can be readily determined by one skilled in the art. It is understood that the precise dosage and duration of treatment is a function of the tissue being treated and may be determined empirically using known routine testing protocols or by extrapolation from in vivo or in vitro test data. It is to be noted that concentrations and dosage values may also vary with the age of the individual treated. It is to be further understood that for any particular subject, specific dosage regimens may be adjusted over time according to the individual need and the professional judgment of the person administering or supervising the administration of the formulations, and that the concentration ranges set forth herein are exemplary only and are not intended to limit the scope or practice of the claimed formulations.

Oral pharmaceutical dosage forms include solids, liquids, and creams or gels. The solid dosage forms are tablets, lozenges, capsules, granules, and bulk powders. Pharmaceutically acceptable carriers utilized in solid doses may include binders, lubricants, diluents, disintegrating agents, coloring agents, flavoring agents, and wetting agents. Solid formulations are administered by placing them in the mouth and allowing dissolution thus facilitating their contact with the mucosal surfaces. The composition may also be incorporated into a chewing gum or other chewable candy.

Liquid oral dosage forms include aqueous solutions, emulsions, suspensions, solutions and/or suspensions reconstituted from non-effervescent granules and effervescent preparations reconstituted from effervescent granules. Aqueous solutions include, for example, elixirs and syrups. Among the preferred oral dosage formulations are mouthwashes containing the capsaicin or capsaicinoid and other active ingredients. Emulsions are either oil-in water or water-in-oil. Elixirs are clear, sweetened, hydroalcoholic preparations. Pharmaceutically acceptable carriers used in elixirs include solvents. An emulsion is a two-phases system in which one liquid is dispersed in the form of small globules throughout another liquid. Pharmaceutically acceptable carriers used in emulsions are non-aqueous liquids, emulsifying agents and preservatives. Suspensions use pharmaceutically acceptable suspending agents and preservatives. Pharmaceutically acceptable substances used in non-effervescent granules, to be reconstituted into a liquid oral dosage form, include diluents, sweeteners and wetting agents. Pharmaceutically acceptable substance used in effervescent granules, to be reconstituted into a liquid oral dosage form, include organic acids and a source of carbon dioxide. Coloring and flavoring agents may be used in all of the above dosage forms. The liquid may be administered by spraying, drinking, or by swishing and expectorating so that it thoroughly contacts the mucosal surfaces of the mouth.

The cream or gel formulations may be water-based or oil-based, and may contain agents to allow them to remain in contact with the area to be treated. The creams or gels are administered by applying them to the mucosal surfaces of the mouth.

In all cases, the preferred method of using these formulations for the treatment of mucositis is to initiate treatment prior to the development of ulcerations of the oral mucosa. Treatment following development of ulceration will provide pain relief but may not offer the same degree of relief as initiating treatment earlier.

The concentration of the pharmaceutically active compound is adjusted so that oral application provides an effective amount to produce the desired pharmacological effect. The exact dose depends on the age, weight and condition of the patient or animal, as is known in the art.

The oral formulations of the compositions of this invention relieve xerostomia when applied to the oral mucosa. The composition may be administered to the affected area as necessary to provide increased production of saliva and relief from the discomfort caused by dry mouth. Relief may be temporary or permanent, and may even be evident after a single dose of the composition as these compounds enhance the production of saliva. The composition may be administered before meals to aid in swallowing and processing food, or before bedtime to minimize formation of caries caused by lack of saliva. The composition should be administered in an amount sufficient to provide relief from xerostomia that is within safety guidelines established by the FDA. Determining the appropriate amount to administer to a particular subject is routine and within the skill of the person of ordinary skill in the art.

Pharmaceutical and cosmetic carriers or vehicles suitable for administration of the oral and topical compositions provided herein include any such carriers known to those skilled in the art to be suitable for the particular mode of administration. To formulate these compositions, a weight fraction of a composition according to this invention is dissolved, suspended, dispersed or otherwise mixed in a selected vehicle at an effective concentration such that the mucositic or xerostomic condition is relieved or ameliorated. Generally, for purposes of oral compositions, a dissolvable solid vehicle or a chewing gum are preferred because they allow the capsaicin to remain in contact with the mucosal surfaces of the mouth for a longer period of time, thereby maximizing the production of saliva.

2. Methods of Treatment

The methods of this invention provide relief from mucositis or xerostomia, and comprise orally administering one of the compositions according to this invention to ameliorate or eliminate dry mouth symptoms. The composition may be administered in a variety of forms, as described above with respect to the various formulations contemplated for use with the compositions of this invention. The method of administration takes into account the particular formulation being used, i.e., orally applying a gel or cream, chewing a piece of gum, allowing a lozenge or ice to dissolve or melt, etc. The methods of treating mucositis and/or xerostomia according to this invention may be used to treat dry mouth caused by, for example, dehydration, use of drugs causing mucositic or xerostomic side effects, radiographic treatment of the head or neck, and various systemic disorders, and any condition that causes dry mouth. The compositions may also be used to prevent development of mucositis or xerostomia in patients considered to be at risk for developing these conditions. It is believed that by treating such patients, salivary function may be better preserved than if no prophylactic treatment is administered.

Compositions for use in treating mucositis, xerostomia, or dry mouth are administered orally, in amounts that alleviate dry mouth in the animal being treated. The compositions may be administered as necessary to achieve the desired result. Again, it is understood that the precise treatment regimen depends upon the individual treated and may be ascertained empirically depending upon the formulation and the age of the treated individual. Any regimen is acceptable as long as the desired anti-xerostomic effects are achieved without substantial deleterious or sustained undesirable side effects.

### EXAMPLES

The compositions and methods for treating mucositis and xerostomia according to this invention will be described in more detail in the following non-limiting example.

### Example 1

### Use of Capsaicin to Induce Salivation

A 49 year old white male subject was used as a test subject to determine whether application of capsaicin to oral mucosa causes salivation. Approximately 0.25 inches of capsaicin (0.025% generic capsaicin cream) was applied to the oral mucosa. Within a few minutes, the subject reported a warm feeling in the mouth, which increased in intensity for about twenty minutes. Saliva was collected for a period of about 15 minutes in a paper cup. The amount was compared to the amount of saliva collected for a period of 15 minutes before applying capsaicin to the oral mucosa. It is estimated that application of capsaicin to the oral mucosa resulted in an increase in salivation of about two to three times the baseline level of salivation. The increased salivation continued for approximately an hour. In preferred embodiments of the present invention, capsaicin or capsaicinoid is combined with one or more of the following: antihistamines such as Benadryl®, or other antihistamines, antibacterials, antifungals, antivirals, and/or antipruritis medications, such as those described above.

Thus, what has been described is a composition and method for treating mucositis, xerostomia, or dry mouth. While the present invention has been described with respect to what are presently considered to be the preferred embodiments, it is to be understood that the invention is not limited to the disclosed embodiments. To the contrary, the invention is intended to cover various modifications and equivalent arrangements included within the spirit and scope of the appended claims. Therefore, the scope of the following claims is to be accorded the broadest interpretation so as to encompass all such modifications and equivalents.

All references set out herein are incorporated by reference.

We claim:

1. A composition for use in treating mucositis, comprising:
capsaicin or capsaicin derivative; and
one or more additional compounds useful in treating mucositis, wherein the composition is provided in an oral delivery vehicle.

2. The composition of claim 1, wherein the capsaicin derivative is selected from the group consisting of cis-capsaicin (civamide), N-phenylmethylalkenamide capsaicin derivatives, dihydrocapsaicin, norhydrocapsaicin, homocapsaicin, homohydrocapsaicin, civamide, nonivamide, olvanil, N-[(4-(2-aminoethoxy)-3-methoxyphenyl)methyl]-9Zoctadecanamide, N-oleyl-homovanillamide, and their analogs and derivatives.

3. The composition of claim 1, wherein the capsaicin or capsaicin derivative is present in an amount of from about 0.01% to about 2.5% by weight.

4. The composition of claim 1, wherein the one or more additional compounds are selected from the group consisting of antifungal compounds, antibacterial compounds, antiviral compounds, anesthetic compounds, analgesic compounds, anti-inflammatory compounds, cytoprotective compounds, anti-histamine compounds, anti-mucositic compounds, cell differentiating agents, and anti-xerostomic compounds.

5. The composition of claim 1, wherein the oral delivery vehicle is selected from the group consisting of lozenges, chewing gums, other candies, creams, gels, and sprays.

6. A method for treating mucositis, comprising the steps of:
a) administering to a patient a therapeutically effective amount of a composition comprising:
i) a capsaicin or capsaicin derivative; and
ii) one or more additional compounds useful in treating mucositis, and
b) repeating administration as necessary to relieve the mucositis.

7. The method of claim 6, wherein the capsaicin derivative is selected from the group consisting of cis-capsaicin (civamide), N-phenylmethylalkenamide capsaicin derivatives, dihydrocapsaicin, norhydrocapsaicin, homocalpsaicin, homohydrocapsaicin, civamide, nonivamide, olvanil, N-[(4-(2-aminoethoxy)-3-methoxyphenyl)methyl]-9Z-octadecanamide, N-oleyl-homovanillamide, and their analogs and derivatives.

8. The method of claim 6, wherein the composition is administered using a delivery system selected from the group consisting of lozenges, chewing gums, creams, gels, and sprays.

9. The method of claim 6, wherein the one or more additional compounds are selected from the group consisting of antifungal compounds, antibacterial compounds, antiviral compounds, anesthetic compounds, analgesic compounds, anti-inflammatory compounds, cytoprotective compounds, anti-histamine compounds, anti-mucositic compounds, and anti-xerostomic compounds.

10. The method of claim 6, wherein the composition is suitable for application to oral mucous membranes.

11. The method of claim 6, wherein the composition is administered to a mammal.

12. The method of claim 11, wherein the mammal is a human being.

13. A method for preparing a composition for use in treating mucositis, comprising the steps of:
a) providing a compound comprising
i) capsaicin or a capsaicin derivative; and
ii) one or more additional compounds selected from the group consisting of antifungal compounds, antibacterial compounds, antiviral compounds, anesthetic compounds, analgesic compounds, anti-inflammatory compounds, cytoprotective compounds, anti-histaminic compounds, anti-mucositic compounds, and anti-xerostomic compounds; and
b) combining said compound with an oral delivery system, thereby forming an oral preparation.
